(19) United States
(12) Patent Application Publication
(10) Pub. No.: US 2025/0134594 A1
(43) Pub. Date: May 1, 2025

(54) INTRAOPERATIVE INTERFACING METHOD FOR COMPUTER-ASSISTED SURGERY SYSTEM

(71) Applicant: ORTHOSOFT ULC, Montreal (CA)

(72) Inventor: Freddie RAMIREZ PRESTON, Montreal (CA)

(21) Appl. No.: 18/931,295

(22) Filed: Oct. 30, 2024

Related U.S. Application Data
(60) Provisional application No. 63/594,283, filed on Oct. 30, 2023.

(51) Int. Cl. A61B 34/00; A61B 34/10; A61B 34/20

(52) U.S. Cl. CPC A61B 34/00; A61B 2034/101; A61B 2034/107; A61B 2034/2046

(57) ABSTRACT

There is disclosed a system for generating a 3D anatomical feature surface. The system comprises a processing unit and a non-transitory computer-readable memory communicatively coupled to the processing unit. The memory comprises computer-readable program instructions executable by the processing unit for tracking a surgical tool configured for contacting an anatomical feature surface, in a first mode, identifying from the tracking a trigger orientation, in response to the trigger orientation being identified, switching the tracking to a second mode in which surfacic data is ready to be recorded from the tracking of the tool, in response to said switching the tracking to the second mode, identifying a signal representative of the surgical tool being in a recording position, in response of the signal being identified, recording surfacic data from the tracking of the tool and generating and outputting a 3D model of the anatomical feature using the surfacic data.

### CROSS-REFERENCE TO RELATED APPLICATION

The present application claims the priority of U.S. Patent Application No. 63/594,283, filed on Oct. 30, 2023, and incorporated herein in its entirety by reference.

### TECHNICAL FIELD

The present application relates to computer-assisted surgery including bone and tool tracking, and to the calibration of instruments in the context of computer-assisted surgery.

### BACKGROUND OF THE ART

Tracking of surgical instruments or tools is an integral part of computer-assisted surgery (hereinafter “CAS”), including robotized CAS. The end effector, the tools, bodily parts are tracked for position and/or orientation in such a way that relative navigation information pertaining to bodily parts is obtained. The information is then used in various interventions (e.g., orthopedic surgery, neurological surgery) with respect to the body, such as bone alterations, implant positioning, incisions and the like during surgery.

When it comes to CAS in which the surgeon operates the patient using tracked tool, there is a challenge in the flow of the procedure when the surgeon wishes to interact with the CAS system, as it may require that the surgeon focus away from the surgical site, and manipulate a mouse or a touch screen, for example. Improving the flow of the procedure when tracking a tool may require a clear distinction between tracking when the tool is used for its purpose, e.g. for painting an anatomical feature, and tracking when the tool is not in operation. Accordingly, some CAS system have integrated automated surgical workflows, by which the CAS system automatically progresses through steps of a surgical workflow, upon determining that a task associated with a step is completed. However, in some instances, the actions of the CAS system may not be aligned with the intentions of the human operator of the system.

There is thus room for improvement.

### SUMMARY

In accordance with a first aspect of the present disclosure, there is provided a system for generating a 3D anatomical feature surface comprising: a processing unit; and a non-transitory computer-readable memory communicatively coupled to the processing unit and comprising computer-readable program instructions executable by the processing unit for: tracking a surgical tool configured for contacting an anatomical feature surface, in a first mode; identifying from the tracking a trigger orientation; in response to the trigger orientation being identified, switching the tracking to a second mode in which surfacic data is ready to be recorded from the tracking of the surgical tool; in response to said switching the tracking to the second mode, identifying a signal representative of the surgical tool being in a recording position; in response of the signal being identified, recording surfacic data from the tracking of the surgical tool; and generating and outputting a 3D model of the anatomical feature using the surfacic data.

Further in accordance with the first aspect, for instance, the trigger orientation has the tool pointing substantially upward.

Still further in accordance with the first aspect, for instance, identifying the signal includes identifying a position and a orientation representative of the surgical tool being in contact with the anatomical feature surface.

Still further in accordance with the first aspect, for instance, identifying the signal includes receiving an input signal provided by a user.

Still further in accordance with the first aspect, for instance, identifying the signal includes identifying a defined period that has elapsed since the identification of the trigger orientation.

Still further in accordance with the first aspect, for instance, said tracking is performed using optical tracking.

Still further in accordance with the first aspect, for instance, tracking the surgical tool configured for contacting an anatomical feature surface includes tracking the surgical tool configured for contacting a bone.

Still further in accordance with the first aspect, for instance, tin response to the trigger orientation being identified, at least one region of at least the anatomical feature surface for which surfacic data is to be recorded is displayed graphically.

Still further in accordance with the first aspect, for instance, displaying graphically the at least one region includes updating a graphical display as surfacic data is recorded for the at least one region.

Still further in accordance with the first aspect, for instance, the trigger orientation programmed in the non-transitory computer-readable memory is retrieved.

Still further in accordance with the first aspect, for instance, a referential system for the anatomical surface feature is defined prior to said tracking.

Still further in accordance with the first aspect, for instance, another trigger orientation is identified from the tracking; in response to the other trigger orientation being identified, switching the tracking to a third mode; in response to said switching the tracking to the third mode, erasing data acquired during the tracking, and identifying a signal representative of the surgical tool being in a recording position; in response of the signal being identified, recording data from the tracking of the tool.

Still further in accordance with the first aspect, for instance, recording surfacic data from the tracking of the surgical tool includes recording the surfacic data from a continuous movement of the surgical tool on the anatomical feature surface.

In accordance with a second aspect, there is provided a system for navigating through a surgical workflow comprising: a processing unit; and a non-transitory computer-readable memory communicatively coupled to the processing unit and comprising computer-readable program instructions executable by the processing unit for: tracking at least one surgical tool configured for recording points on an anatomical feature surface as the surgical tool contacts the anatomical feature surface, in a first moment of the surgical workflow; identifying from the tracking a trigger orientation; in response to the trigger orientation being identified, switching the surgical workflow to a second moment thereof; and in response to said switching the tracking to the second moment, tracking the at least one surgical tool in accordance with the second moment of the surgical workflow.

Further in accordance with the second aspect, for instance, switching to the second moment of the surgical workflow includes switching to a moment of the surgical workflow that is before the first moment.

Still further in accordance with the second aspect, for instance, switching to the second moment of the surgical workflow includes deleting at least some of the points recorded in the first moment.

Still further in accordance with the second aspect, for instance, switching to the second moment of the surgical workflow includes prolonging an action associated with the first moment of the surgical workflow.

Still further in accordance with the second aspect, for instance, the trigger orientation has the tool pointing substantially upward.

Still further in accordance with the second aspect, for instance, a referential system for the anatomical surface feature is defined prior to said tracking.

Still further in accordance with the second aspect, for instance, the trigger orientation programmed in the non-transitory computer-readable memory is retrieved.

### DESCRIPTION OF THE DRAWINGS

FIG. 1 is a schematic view of a robotic surgery system in accordance with an aspect of the present disclosure, relative to a patient;

FIG. 2 is a block diagram of the tracking system for robotized computer-assisted surgery of FIG. 1;

FIG. 3 is a front view of a tracker device in a trigger orientation configuration; and

FIG. 4 is a flow chart of a method for tracking a tracker device with a robotic surgery system.

### DETAILED DESCRIPTION

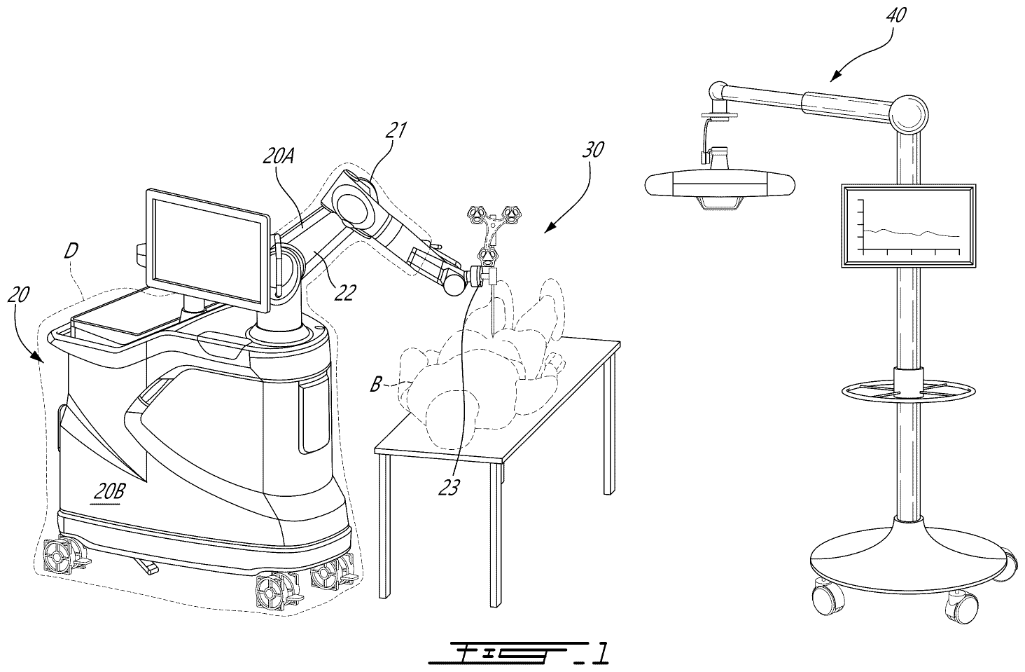

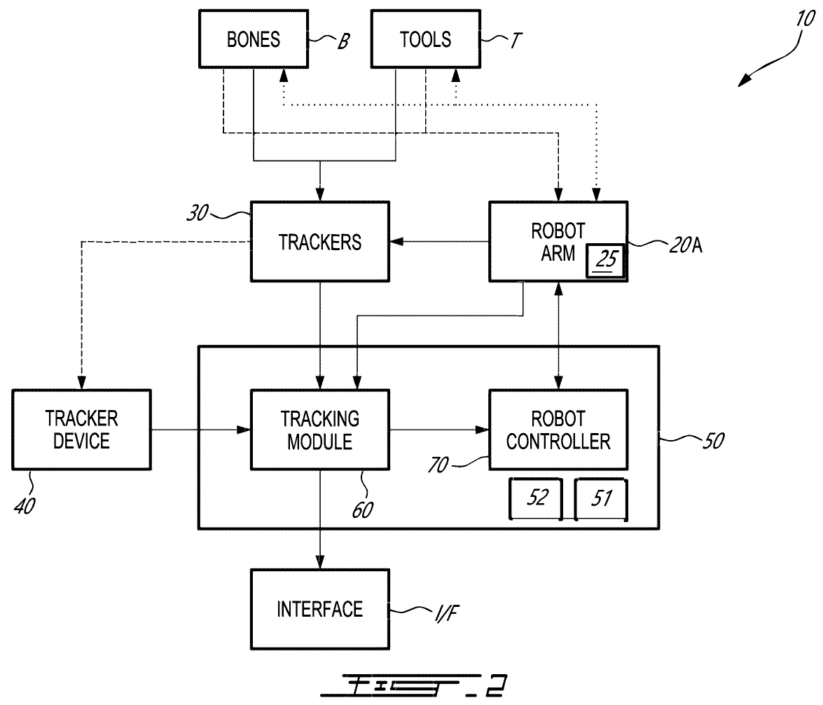

Referring to FIGS. 1 and 2, a robotic surgery system for computer-assisted surgery (CAS) system is generally shown at 10, and is used to provide surgery assistance to an operator. For simplicity, it will be referred to herein as the system 10. In FIG. 1, the system 10 is shown relative to a dummy patient in prone decubitus, but only as an example. The system 10 could be used for any body parts, including non-exhaustively hip joint, spine, and shoulder bones, for orthopedic surgery, but could also be used in other types of surgery. For example, the system 10 could be used for surgery of all sorts, such as brain surgery, and soft tissue surgery.

The robotic surgery system 10 may be robotized in a variant, and has, may have or may be used with a robot 20, optical trackers 30, a tracker device 40, a CAS controller 50 (also known as a super controller 50), a tracking module 60, and a robot controller 70 (also known as a robot driver), or any combination thereof:
The robot 20, shown by its robot arm 20A may optionally be present as the working end of the system 10, and may be used to perform or guide bone alterations as planned by an operator and/or the CAS controller 50 and as controlled by the CAS controller 50. The robot arm 20A may also be configured for collaborative/cooperative mode in which the operator may manipulate the robot arm 20A, or the tool supported by the robot arm 20, though the tool may be operated by a human operator. For example, the tooling end, also known as end effector, may be manipulated by the operator while supported by the robot arm 20A. The robot 20 may be the coordinate measuring machine (CMM) of the robotic surgery system 10;
The optical trackers 30 are positioned on the robot 20, on patient tissue (e.g., bones B), and/or on the tool(s) T and surgical instruments, and provide tracking data for the robot 20, the patient and/or tools.
The tracking device 40, also known as a sensor device, apparatus, etc performs optical tracking of the optical trackers 30, so as to enable the tracking in space (a.k.a., navigation) of the robot 20, the patient and/or tools;
The CAS controller 50, also known as the super controller, includes the processor(s) and appropriate hardware and software to run a computer-assisted surgery procedure in accordance with one or more workflows. The CAS controller 50 may include or operate the tracking device 40, the tracking module 60, and/or the robot controller 70. As described hereinafter, the CAS controller 50 may also drive the robot arm 20A through a planned surgical procedure;
The tracking module 60 is tasked with determining the position and/or orientation of the various relevant objects during the surgery procedure, such as the end effector of the robot arm 20A, bone(s) B and tool(s) T, using data acquired by the tracking device 40 and by the robot 20, and/or obtained from the robot controller 70. The position and/or orientation may be used by the CAS controller 50 to control the robot arm 20A;
The robot controller 70 is tasked with powering or controlling the various joints of the robot arm 20A, based on operator demands or on surgery planning, and may also be referred to as a robot controller module that is part of the super controller 50. The robot controller 70 may also optionally calculate robot movements of the robot arm 20A, so as to control movements of the robot arm 20A autonomously in some instances, i.e., without intervention from the CAS controller 50;
An additional camera(s) may be present, for instance as a complementary registration tool. The camera may for instance be mounted on the robot 20, such as on the robot arm 20A, such that the point of view of the camera is known in the frame of reference, also known as the coordinate system.

Other components, devices, systems, may be present, such as surgical instruments and tools T, interfaces I/F such as displays, screens, computer station, servers, and like etc. The interfaces I/F may include a mouse and/or a foot pedal that may be used as a clicking device. Secondary tracking systems may also be used for redundancy.

Referring to FIG. 1, the robot 20 may have the robot arm 20A stand from a base 20B, for instance in a fixed relation relative to the operating-room (OR) table supporting the patient, whether it is attached or detached from the table. The robot arm 20A has a plurality of joints 21 and links 22, of any appropriate form, to support an end effector 23 that may interface with the patient, or may be used during surgery without interfacing with the patient. For example, the end effector or tool head may optionally incorporate a force/torque sensor for collaborative/cooperative control mode, in which an operator manipulates the robot arm 20A. The robot arm 20A is shown being a serial mechanism, arranged for the tool head 23 to be displaceable in a desired number of degrees of freedom (DOF). The tool head 23 may for example be a support that is not actuated, the support being used to support a tool, with the robot arm 20A used to position the tool relative to the patient. In a variant, the robot arm 20A controls 6-DOF movements of the tool head, i.e., X, Y, Z in the coordinate system, and pitch, roll and yaw. Fewer or additional DOFs may be present. For simplicity, only a fragmented illustration of the joints 21 and links 22 is provided, but more joints 21 of different types may be present to move the end effector 23 in the manner described above. The joints 21 are powered for the robot arm 20A to move as controlled by the CAS controller 50 in the six DOFs, and in such a way that the position and orientation of the end effector 23 in the coordinate system may be known, for instance by readings from encoders on the various joints 21. Therefore, the powering of the joints is such that the end effector 23 of the robot arm 20A may execute precise movements, such as moving along a single direction in one translation DOF, or being restricted to moving along a plane, among possibilities. Such robot arms 20A are known, for instance as described in U.S. patent application Ser. No. 11/610,728, and incorporated herein by reference.

The end effector 23 of robot arm 20A may be defined by a chuck or like tool interface, typically actuatable in rotation. As a non-exhaustive example, numerous tools may be used as end effector for the robot arm 20A, such tools including a registration pointer as shown in FIG. 1, equipped with a tracker device 30, a reamer (e.g., cylindrical, tapered), a reciprocating saw, a retractor, a camera, an ultrasound unit, a laser rangefinder or light-emitting device (e.g., the indicator device of U.S. Pat. No. 8,882,777), a laminar spreader, an instrument holder, or a cutting guide, depending on the nature of the surgery. The various tools may be part of a multi-mandible configuration or may be interchangeable, whether with human assistance, or as an automated process. The installation of a tool in the tool head may then require some calibration in order to track the installed tool in the X, Y, Z coordinate system of the robot arm 20A.

The end effector 23 of the robot arm 20A may be positioned by the robot 20 relative to surgical area A in a desired orientation according to a surgical plan, such as a plan based on preoperative imaging. Due to the proximity between the robot 20 and the surgical area A, the robot 20 may be covered partially with a surgical drape D, also known as a surgical robotic drape. The surgical drape D is a sterile panel (or panels), tubes, bags or the like that form(s) a physical barrier between the sterile zone (e.g., surgical area) and some equipment that may not fully comply with sterilization standards, such as the robot 20. In an embodiment, the surgical drape D is transparent such that one can see through the drape D. In an embodiment, the robot is entirely covered with the surgical drape D, and this includes the base 20B, but with the exception of the end effector 23. Indeed, as the end effector 23 interacts or may interact with the human body, it may be sterilized and may not need to be covered by the surgical drape D, to access the patient. Some part of the robot 20 may also be on the sterile side of the surgical drape D. In a variant, a portion of the robot arm 20A is covered by the surgical drape D. For example, the surgical drape D may be in accordance with U.S. patent application Ser. No. 15/803,247, filed on Nov. 3, 2017 and incorporated herein by reference.

In order to position the end effector 23 of the robot arm 20A relative to the patient B, the CAS controller 50 can manipulate the robot arm 20A automatically (without human intervention), or by a surgeon manually operating the robot arm 20A (e.g. physically manipulating, via a remote controller through the interface I/F) to move the end effector 23 of the robot arm 20A to the desired location, e.g., a location called for by a surgical plan to align an instrument relative to the anatomy. Once aligned, a step of a surgical procedure can be performed, such as by using the end effector 23. To assist in the maneuvering and navigating of the robot arm 20A, a tracker device 30 may optionally be secured to the distalmost link, and may be distinct from the tracker device 30 on the instrument supported by the end effector 23.

As shown in FIG. 2, the robot arm 20A may include sensors 25 in its various joints 21 and links 22. The sensors 25 may be of any appropriate type, such as rotary encoders, optical sensors, position switches that are a non-exhaustive list of potential sensors, for the position and orientation of the end effector, and of the tool in the end effector 23 to be known. More particularly, the tracking module 60 may determine the position and orientation of the robot 20 in a frame of reference of the robot 20, such as by obtaining the position (x,y,z) and orientation (phi, theta, ro) of the end effector 23 from the CAS controller 50 using the sensors 25 in the robot arm 20A, i.e., robot coordinates may be an integrated function of the robot 20 in that it may determine the position and orientation of its end effector 23 with respect to its coordinate system. Using the data from the sensors 25, the robot 20 may be the coordinate measuring machine (CMM) of the robotic surgery system 10, with a frame of reference (e.g., coordinate system, referential system) of the procedure being relative to the fixed position of the base 20B of the robot 20. The sensors 25 must provide the precision and accuracy appropriate for surgical procedures. The coupling of tools to the robot arm 20A may automatically cause a registration of the position and orientation of the tools in the frame of reference of the robot 20, though steps of calibration could be performed, as explained further below.

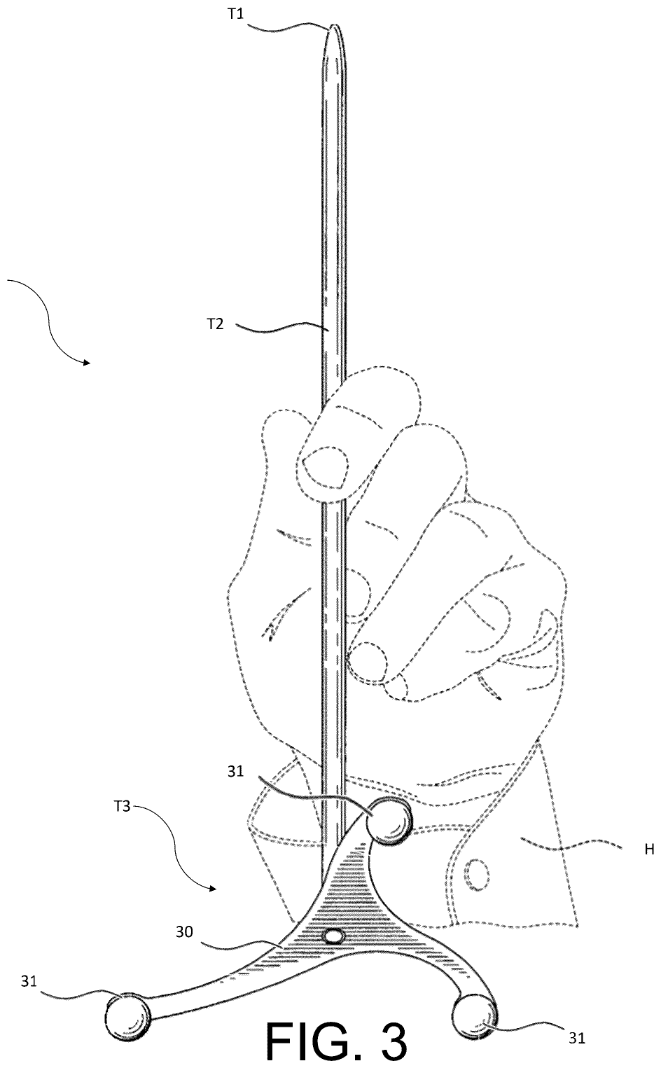

Now referring to FIG. 3, there is shown a front view of a surgical tool T in an exemplary trigger orientation configuration. The surgical tool T is depicted as a registration pointer having a tip T1 at a working end of an elongated rod T2. A handle T3 may be at the opposite end. A tracker 30 may be attached to an end of the elongated rod T2. In the embodiment depicted in FIG. 3, the tracker 30 has three tracker members 31 arranged in a configuration programmed in the CAS controller 50, such as in a scalene triangle, as an example among others. The tracker members 31 are attached to respective arms that are spirally extending outwardly from the center of the tracker 30, the center being attached to the tool T. This is merely a configuration among others. The tracker members 31 may have a sphere shape. It should be however understood that the number, the size and the shape of the tracker members 31 and the configuration of the tracker 30 may vary according to the embodiment. For example, FIG. 1 shows another type of tracker 30, and may be as described in U.S. Pat. No. 8,386,022, incorporated herein by reference. Moreover, while the tool T is illustrated in FIG. 3 as being a registration pointer, other tools may be used in accordance with the present disclosure.

In the depicted embodiment of FIG. 3, the surgical tool T is held in an operator's hand H in a trigger orientation. The trigger orientation has the surgical tool T pointing substantially upward, such that the elongated rod T2 is vertical, i.e., substantially orthogonal to the plane of the floor/ceiling and the tracker 30 is positioned beneath the tip T1. Such trigger orientation may be preferred as it is an orientation in which the tool T is unlikely to be used during a surgery, while performing actions on the body. For example, in a surgery in which the bone of the knee is being modelled, it is unlikely that the surgical tool T will be held substantially vertical and upside down as in the trigger orientation depicted in FIG. 3. As such, the substantially upward and upside down orientation of the surgical tool T may suitably be used only when the surgeon or other operator or user wishes to produce the trigger orientation. It should be understood however that any other suitable trigger orientation of the surgical tool T may be programmed in the CAS controller 50. While the surgical tool T is held in the operator's hand H in the embodiment depicted in FIG. 3, the surgical tool T may also be attached to the robot arm 20A of the robot 20 shown in FIGS. 1 and 2, when the trigger orientation is selected.

Referring to FIG. 2, the CAS controller 50 is shown in greater detail relative to the other components of the robotic surgery system 10. The CAS controller 50 has a processor unit 51 and a non-transitory computer-readable memory 52 communicatively coupled to the processing unit 51 and configured for executing computer-readable program instructions executable by the processing unit 51 to perform some functions, such as tracking the patient tissue and tools, using the position and orientation data from the robot 20 and the readings from the tracker device 40. Accordingly, as part of the operation of the CAS controller 50, the computer-readable program instructions may include an operating system that may be viewed by a user or operator as a GUI on one or more of the interfaces of the robotic surgery system 10. It is via this or these interfaces that the user or operator may interface with the robotic surgery system, be guided by a surgical workflow, obtain navigation data, etc. In a variant, the surgical workflow has automated steps, with the CAS controller 50 processing through the steps in an automated way. For example, through the gathering of data during the surgical procedure, the CAS controller 50 may switch from one step to another in the surgical workflow, after determining that the task associated with the earlier step is completed. The automation of workflow progress is convenient in that the operator (e.g., surgeon) may not have to send a command to the CAS controller 50, which command may require to manipulate an interface, move one's attention away from the surgical site, put temporarily away a tool, etc. The CAS controller 50 may also control the movement of the robot arm 20A via the robot controller module 70. The robotic surgery system 10 may comprise various types of interfaces I/F, for the information to be provided to the operator. For example, a foot pedal is commonly used as part of a computer-assisted surgery system, such as the robotic surgery system 10, as the equivalent of a clicking device (i.e., the buttons on a mouse). The interfaces I/F may include and/or screens including wireless portable devices (e.g., phones, tablets), audio guidance, LED displays, head-mounted display for virtual reality, augmented reality, mixed reality, among many other possibilities. For example, the interface I/F comprises a graphic-user interface (GUI) operated by the system 10. The CAS controller 50 may also display images captured pre-operatively, or using cameras associated with the procedure (e.g., 3D camera, laparoscopic cameras, tool mounted cameras), for instance to be used in the collaborative/cooperative control mode of the system 10, or for visual supervision by the operator of the system 10, with augmented reality for example. The CAS controller 50 may drive the robot arm 20A, in performing the surgical procedure based on the surgery planning achieved pre-operatively, or in maintaining a given position and orientation to support a tool. The CAS controller 50 may run various modules, in the form of algorithms, code, non-transient executable instructions, etc, in order to operate the robotic surgery system 10 in the manner described herein. The CAS controller 50 may be part of any suitable processor unit, such as a personal computer or computers including laptops and desktops, tablets, server, etc.

The tracking module 60 may be a subpart of the CAS controller 50, or an independent module or system. The tracking module 60 receives the position and orientation data from the robot 20 and the readings from the tracker device 40. The tracking module 60 may hence determine the relative position of the objects relative to the robot arm 20A in a manner described below. The tracking module 60 may also be provided with models of the objects to be tracked. For example, the tracking module 60 may track bones and tools, and hence may use virtual bone models and tool models. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc). The bone models may therefore be patient specific. It is also considered to obtain bone models from a bone model library, with the data obtained from the video images used to match a generated 3D surface of the bone with a bone from the bone atlas. The virtual tool models may be provided by the tool manufacturer, or may also be generated in any appropriate way so as to be a virtual 3D representation of the tool(s).

Additional data may also be available, such as tool orientation (e.g., axis data and geometry). By having access to bone and tool models, the tracking module 60 may obtain additional information, such as the axes related to bones or tools.

Still referring to FIG. 2, the CAS controller 50 may have the robot controller 70 integrated therein. However, the robot controller 70 may be physically separated from the CAS controller 50, for instance by being integrated into the robot 20 (e.g., in the robot base 20B). The robot controller 70 is tasked with powering and/or controlling the various joints of the robot arm 20A. The robot controller 70 may also optionally calculate robot movements of the robot arm 20A, so as to control movements of the robot arm 20A autonomously in some instances, i.e., without intervention from the CAS controller 50. There may be some force feedback provided by the robot arm 20A to avoid damaging the bones, to avoid impacting other parts of the patient or equipment and/or personnel. The robot controller 70 may perform actions based on a surgery planning. The surgery planning may be a module programmed specifically for any given patient, according to the parameters of surgery desired by an operator such as an engineer and/or surgeon. The parameters may include geometry of selected, planned bone cuts, planned cut depths, sequence or workflow of alterations with a sequence of surgical steps and tools, tools used, etc.

As observed herein, the trackers 30 and the tracker device 40 may be complementary tracking technology. The position and orientation of the surgical tool T calculated by the tracking module 60 using optical tracking may be redundant over the tracking data provided by the robot controller 70 and/or the CAS controller 50 and its embedded robot arm sensors 25, referred to as maneuvering data for the robot arm 20A. However, the redundancy may assist in ensuring the accuracy of the tracking of the surgical tool T, and end effector 23. More particularly, the combination of the navigation data from the tracker device 40 and that from robot controller 70 may strategically be used to improve the accuracy of the calibration of the instruments T with their trackers 30. The present system 10 and related method may apply to the instruments T with trackers 30 as in FIG. 3, but also to other types of trackers 30, such as retroreflective spheres, QR codes, etc.

Consequently, the tracking module 60 may combine the optical tracking data from the tracker device 40 to the position and orientation data from the sensors 25 embedded in the robot arm 20A, for the positional tracking data for the objects may be calculated by the tracking module 60, as detailed below. Therefore, the combination by the tracking module 60 of the tracking from the robot arm 20A and that from the tracker device 40 enable the tracking module 60 to track objects with a continuous and robust navigation data.

Again, the distinct sources of tracking data, e.g., the embedded tracking from the sensors 25 in the robot arm 20A, and optical tracking using the robot base 20A, and other trackers 30, ensure that sufficient tracking data is available for the tracking module 60 (FIG. 2) to determine a position of the bone B, of the tool T and of the end effector 23 in the frame of reference. The tracking module 60 may adjust the readings if movement is detected for the tracker device 40, with the configuration of the robotic surgery system 10.

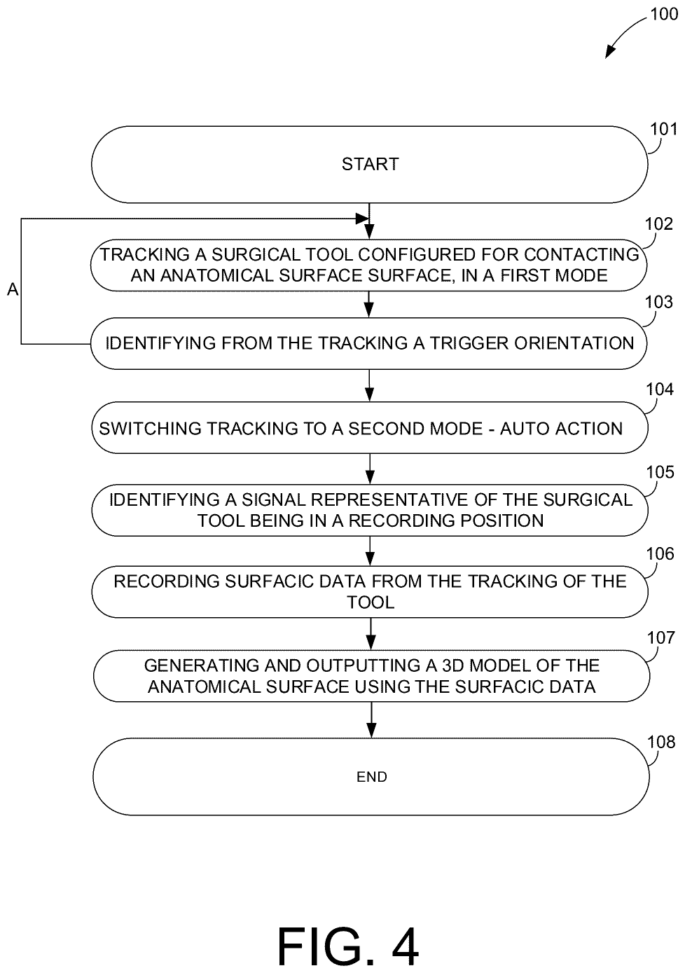

In an embodiment, the tracking module 60 uses a tracker 30 on the bone B or other body portion or OR table to obtain the orientation of the bone B in the coordinate system, and locates the bone B using other methods, such as obtaining the position and orientation of a probing tool using the encoders in the robot arm 20A, in a registration procedure described below. Stated differently, the bone B may be fixed on the OR table and the system 10 may rely on trackers 30 fixed to the OR table to optically track the bone B. Now that the various components of the robotic surgery system 10 have been described, a contemplated procedure performed with the robotic surgery system 10 or with a similar CAS system is set forth, with reference to a flow chart illustrative of a method 100 for tracking an end effector of a robot in computer-assisted surgery is shown at in FIG. 4, and is an example of a procedure that may be performed by the CAS controller 50 and/or other parts of the robotic surgery system 10 of the present disclosure. For example, the method 100, may be computer-readable program instructions in the non-transitory computer-readable memory 52 for example, and executable by the processing unit 51 communicatively coupled to the processing unit 51.

Now referring to FIG. 4, the method 100 starts at step 101. The method may be referred to as a method for tracking or navigating a tool and/or anatomic feature or surface thereof in computer-assisted surgery, a method for generating a 3D anatomic feature surface, a method for navigating through a surgical workflow. It should be appreciated that the method 100 may be implemented using the CAS controller 50 of the system 10. Moreover, as the method 100 is used in the context of surgery, the method 100 must be executed in realtime or quasi realtime, i.e., at a frequency by which any computing delay is not or minimally observable by a user. The method 100 may be used to generate a 3D model, that may be used in a graphical display, and this may occur intraoperatively, within accuracy and precision thresholds expected in surgery. Thus, the step 101 may happen intraoperatively, at or after the beginning of the surgery, etc. At step 102, a surgical tool configured for contacting a surface of an anatomical feature is tracked, in a first mode. The tracking may be performed using the tracking device 40 and/or the sensors 25 presented above, and may include the tracking of the tracker 30, or any other suitable tracker. For example, the tracking may be done using depth camera systems, such that 3D trackers such as those shown in FIG. 3, may not be required. In the first mode, the system 10 is configured for tracking the position and/or orientation of the tool T. Generally, the position and the orientation are not recorded in the first mode, though they may be depending on the nature of the surgery. For example, the first mode includes recording points on a surface of a bone, calibrating the tool T, etc. As an example, the tool T is a registration pointer as shown in FIG. 3. The registration pointer is used along with an interface I/F, such as a foot pedal or the like, to record the position of various points in a frame of reference. This is conventionally but non-exclusively performed in minimally invasive surgery or surgical procedures involving smaller incisions, when visibility of a surface is limited because of voluntarily limited incisions to soft tissue, and/or the concave nature of the aspects whose surfaces must be digitized (e.g., glenoid and other parts of scapula, acetabulum). According to one possible way of recording points, the user positions the tool against the surface of the bone, and then indicates that recording may occur, in a snapshot manner. One known way to give the indication is to user an interface I/F such as a foot pedal, just like clicking a mouse. A mouse, a touchscreen could also be used, as manipulated by someone else or by the operator, provided the tool remains in contact with the surface. Accordingly, a cloud of points may be gathered. Another way of recording points is known as painting. In painting, the user maintains a continuous contact between tool and surface to be digitized. Again, an interface I/F is used to start and stop the painting action. For example, a foot pedal may be conveniently used, with the user keeping a button depressed during all of the painting recording, in similar fashion to a drag and drop action. Alternatively, the button may be used as an on/off button. The system may be configured based on the user's preference. Other interfaces may be used in the two scenarios described above.

Prior to step 102, the anatomical feature surface or a reference thereof may be mapped using trackers 30 attached to the patient or using a 3D camera. The mapping may include obtaining a 3D surface of the anatomical feature, or sufficient points on the anatomical feature to create a reference system (e.g., a plane, an axis). Such mapping may serve as a calibration in which the relative position and orientation of the tool T may be obtained with respect to the anatomical feature surface or the referential system. The mapping may be reperformed during the surgery should the patient's position changes, though the change may be captured by a tracker 30 secured to the anatomical feature, or should a different anatomical feature surface need to be modeled. The mapping of step 102 may be referred to as a frame of reference, a coordinate system, a referential frame, etc. It may or may not include a tracker (e.g., tracker 30) on a bone or on structure immovable relative to the bone. In some instances, the bone does not move and may not have any tracker 30 associated with it.

In a variant, in step 102, the controller 50 determines that the task associated with the first mode has been completed. For example, the controller 50 may determine that the number of points in a point recording is sufficient, and/or that the surface painted covers a sufficient surface area. The actions of the controller 50 may be based on various criteria: number of points, surface area covered, distance covered, etc. The criteria may be primarily quantitative in nature, and may involve thresholds, etc. In accordance with the surgical workflow, the controller 50 may be configured to automatically switch to a next step, such as the recording of surface points of another surface, or other subsequent steps in the surgical procedure (e.g., resection, etc).

However, while the controller 50 may have moved on to a subsequent step, e.g., automatically, the operator (e.g., surgeon) may not be satisfied with the information gathered. For example, the surfacic data obtained may satisfy pre-established quantitative thresholds, but may not comply with the qualitative expectations from the operator. Because the controller 50 has moved on to another step, the operator may have to interact with the controller 50 to return to the previous step, and this may cause a disruption in the maneuvers of the operator (e.g., putting the tool aside, moving away from surgical site, removing gloves, touching a touchscreen, etc). If such is the case, the operator may use the step 103 to refuse the controller 50's automated action in the surgical workflow. Likewise, the operator may be dissatisfied with the data acquired, and may want to delete it. Stated differently, the operator may want to return to the previous step in a surgical workflow. Therefore, the trigger orientation and step 103 may be used for any of these reasons, among others, and this is shown at A.

At step 103, a trigger orientation is identified from the tracking. The trigger orientation may be programmed in the non-transitory computer-readable memory. It should be understood that the trigger orientation may be retrieved from other processing devices such as a server coupled to the controller 50 of the system 10. The trigger orientation may be the orientation represented in FIG. 3. In response to the trigger orientation being identified via image processing by the controller 50, the tracking may return to the prior step 102, to add on to the tracking of the tool in the first mode, or to erase data acquired, etc. For example, this allows the operator to gather supplemental surfacic data, or reset and reacquire surfacic data. The action that will be automated upon return to step 102 may be configured by the user. Moreover, there may be more than one trigger orientation, with each trigger orientation associated with a given action. For example, one trigger orientation would lead to an “add surfacic data” function, another trigger orientation would lead to an “erase” function, another trigger orientation could lead to an overview of the total acquire surfacic data, indicating where surfacic data still needs to be acquired. For example, another trigger orientation could have the pointer pointing at 45 degrees from horizontal to the right, another at 45 degrees from horizontal to the left. It may be required for the trigger orientation to be held for a given amount of time (e.g., 2 seconds). The trigger orientation may also be dynamic, such as for example the pointer pointing upward, with the pointer moved in an oscillating fashion up and down, as an example among others.

As another possibility, the trigger orientation may be used in another manner, exemplified by step 104. At step 104, in response to the trigger orientation being identified, the tracking is switched to a second mode in which surfacic data is ready to be recorded from the tracking of the tool, in a scenario in which the first mode is not for recording surfacic data. In the second mode, as compared to the first mode in which the tracking system only tracks the surgical tool T in order to identify the trigger orientation, or tracks the surgical tool T to perform actions differing from the actions in the second mode (e.g., point per point tracking), the tracking system is on standby until a signal representative of the surgical tool being in a recording position is identified. Further, in the second mode, continuous recording of surfacic data is possible (i.e., painting), as opposed to the first mode (point per point recording). In some embodiments, the surfacic data is representative of the position and orientation of working end of the surgical tool. The switch of 104 may be accompanied by the display of data to the user. In a variant, when a switch as in 104 is made, the system may automatically provide a graphical display of all regions for which surfacic data must be acquired in the second mode (shown as auto action in 104). This may include surfacic data of two different regions of a same bone, surfacic data on two or more different bones, cartilage, etc. The display may be updated, such as in realtime, for the user to see her/his progress in the surgical workflow. If there are two trigger orientations, there may a second mode, and a third mode, etc.

It should be understood that upon the identification of the trigger orientation, the surgical tool may not be in position to perform its role during the surgery, for example as it may be upside down as in FIG. 3. As such, the position and orientation of the surgical tool during this period may not be recorded until the surgical tool is in position for performing its role. In one embodiment, the position suited for surgical operation for the surgical tool is having a tip T1 being in contact with the anatomical feature of the patient. This may require that the anatomic feature be tracked (e.g., it may have a referential system), such as by a tracker 30, and that the zone to be painted is known and located in the reference system. When the controller 50 detects a trigger orientation in 103 and activates a switch as in 104, the system acts automatically to lead the surgical workflow to another step, whether it be a mode with a different capture of data, a step back in the workflow, a display of information, or like other automatic action (auto action in 104). This may be referred to as a first moment of the surgical workflow, and a second moment of the surgical workflow. Hence, the tool T used for the trigger orientation is not used as a mouse once the trigger orientation is detected, and switching occurs. The trigger orientation may allow a forward to a backtracking within the surgical workflow.

At step 105, in response to said switching the tracking to the second mode, a signal representative of the surgical tool being in a recording position is identified. The signal may be obtained from the tracking system or not, according to the embodiment. In some embodiments, the signal includes an identification by the tracking system of a position and a orientation representative of the surgical tool being in contact with the anatomical feature surface, such as using the referential system. In some other embodiments, the signal includes an input signal provided by a user, such as via the foot pedal. Such input signal may also be representative of a voice sequence, e.g. “start recording” or “begin recording”, obtained from a voice recognition software, a signal obtained from a physical button pushed by the surgeon and the like. In some other embodiments, the signal includes an identification of a defined period that has elapsed since the identification of the trigger orientation. For example, upon identification of the trigger orientation, a timer of two seconds (or any amount of time needed for the surgeon to get into position) may be started, after which the signal is identified. In a variant, the trigger signal may be a duration of time during which the tool is immobile, after being displaced from the trigger orientation.

At step 106, in response of the signal being identified, surfacic data is recorded from the tracking of the tool. In operation, the surgical tool may be held in the hand of a surgeon or attached to a robot arm. The surgical tool may be used to paint (i.e. generating a 3D model) an anatomical feature (e.g. a bone) by tracking a plurality of position and orientation of the tool via the tracker thereon (or other tracking technology), such as continuously (i.e., uninterrupted period), plurality of position and orientation representative of surfacic data of the anatomical feature. By calculating the distance between the tip T1 of the elongated rod T2 in one embodiment and the tracker 30, e.g. by triangulation, a set of coordinates may be obtained.

In some embodiments, markers may be placed near the surgery region in order to track the position and the orientation of the anatomical feature subject to the surgery, therefore obtaining surfacic data relative to the position and orientation of the anatomical feature (including a referential system). Thus, it is possible to identify when the tool is in contact with the anatomical feature, in which case only the surfacic data obtained from the tool when the latter is in contact with the anatomical feature may be recorded.

In some embodiments, the surfacic data may be recorded periodically, i.e. a set of coordinates is generated after a defined amount of time. In some other embodiments, the set of coordinates may be recorded when the position and the orientation of the tool varies by a defined value. In this case, a model of the anatomical feature may be obtained irrespective of the speed of which the surgeon moves the tool, which may increase the resolution of the model and minimize the size of the model in terms of quantity of data.

While the second mode is described as being relative to tracking and the subsequent steps are for recording surfacic data, the second mode may alternatively be a step out of order in the surgical workflow. For example, the trigger orientation described in 103 may be used by the operator to deviate from the proposed next step of the surgical workflow, to go instead to a different step. In an embodiment, the trigger orientation of step 103 is used for the operator to reach another mode (i.e., the second mode) that consists in another action, such as obtaining different views on screen, taking a pause, switching tracking tools, zooming in, etc. As yet another possibility, the trigger orientation of step 103 may be used for the operator to accept a proposal from the controller 50. For instance, the operator may be queried “data gathering complete” ?. In response, the operator may use the tool and trigger orientation to acquiesce, with or without the assistance of the foot pedal.

At step 107, a 3D model of the anatomical feature is generated and outputted using the surfacic data. The 3D model may be generated using a software that creates a 3D mesh using the recorded set of data. In some cases, a reference model (a magnetic resonance imaging (MRI) scan) may be used as a starting point.

In some embodiments, the recording may stop when a stop signal, such as a voice signal, a signal obtained from a physical button (e.g., foot pedal) or a signal representative of the tool no longer being in contact with the anatomical feature and the like, is identified.

In some embodiments, the tracking may be switched from the second mode to the first mode upon identification of the trigger orientation when the tracking is already in the second mode. In this case, the switching from the second mode to the first mode may be processed after the 3D model has been obtained.

At step 108, the method ends.

The present disclosure may therefore pertain to a system for generating a 3D anatomical feature surface, and may have: a processing unit; and a non-transitory computer-readable memory communicatively coupled to the processing unit and comprising computer-readable program instructions executable by the processing unit for: tracking a surgical tool configured for contacting an anatomical feature surface, in a first mode; identifying from the tracking a trigger orientation; in response to the trigger orientation being identified, switching the tracking to a second mode in which surfacic data is ready to be recorded from the tracking of the surgical tool; in response to said switching the tracking to the second mode, identifying a signal representative of the surgical tool being in a recording position; in response of the signal being identified, recording surfacic data from the tracking of the surgical tool; and generating and outputting a 3D model of the anatomical feature using the surfacic data.

The present disclosure may also relate to a system for navigating through a surgical workflow that may include: a processing unit; and a non-transitory computer-readable memory communicatively coupled to the processing unit and comprising computer-readable program instructions executable by the processing unit for: tracking at least one surgical tool configured for recording points on an anatomical feature surface as the surgical tool contacts the anatomical feature surface, in a first moment of the surgical workflow; identifying from the tracking a trigger orientation; in response to the trigger orientation being identified, switching the surgical workflow to a second moment thereof; and in response to said switching the tracking to the second moment, tracking the at least one surgical tool in accordance with the second moment of the surgical workflow.

The term “communicatively connected” or “communicatively coupled to” may include both direct coupling (in which two elements that are coupled to each other contact each other) and indirect coupling (in which at least one additional element is located between the two elements). For instance, the term “communicatively connected” or “communicatively coupled to” may include a wireless connection over a communication network such as the Internet.

While illustrated in the block diagram of FIG. 2 as groups of discrete components communicating with each other via distinct data signal connections, it will be understood by those skilled in the art that the preferred embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present preferred embodiment. The embodiments of the technology described above are intended to be exemplary only. The scope of the invention is therefore intended to be limited solely by the scope of the appended claims.

1. A system for generating a 3D anatomical feature surface comprising:
a processing unit; and
a non-transitory computer-readable memory communicatively coupled to the processing unit and comprising computer-readable program instructions executable by the processing unit for:
tracking a surgical tool configured for contacting an anatomical feature surface, in a first mode;
identifying from the tracking a trigger orientation;
in response to the trigger orientation being identified, switching the tracking to a second mode in which surfacic data is ready to be recorded from the tracking of the surgical tool;
in response to said switching the tracking to the second mode, identifying a signal representative of the surgical tool being in a recording position;
in response of the signal being identified, recording surfacic data from the tracking of the surgical tool; and
generating and outputting a 3D model of the anatomical feature using the surfacic data.

2. The system according to claim 1, wherein the trigger orientation has the tool pointing substantially upward.

3. The system according to claim 1, wherein identifying the signal includes identifying a position and a orientation representative of the surgical tool being in contact with the anatomical feature surface.

4. The system according to claim 1, wherein identifying the signal includes receiving an input signal provided by a user.

5. The system according to claim 1, wherein identifying the signal includes identifying a defined period that has elapsed since the identification of the trigger orientation.

6. The system according to claim 1, wherein said tracking is performed using optical tracking.

7. The system according to claim 1, wherein tracking the surgical tool configured for contacting an anatomical feature surface includes tracking the surgical tool configured for contacting a bone.

8. The system according to claim 1, further including, in response to the trigger orientation being identified, displaying graphically at least one region of at least the anatomical feature surface for which surfacic data is to be recorded.

9. The system according to claim 8, wherein displaying graphically the at least one region includes updating a graphical display as surfacic data is recorded for the at least one region.

10. The system according to claim 1, including retrieving the trigger orientation programmed in the non-transitory computer-readable memory.

11. The system according to claim 1, including defining a referential system for the anatomical surface feature prior to said tracking.

12. The system according to claim 1, further including identifying from the tracking another trigger orientation;
in response to the other trigger orientation being identified, switching the tracking to a third mode;
in response to said switching the tracking to the third mode, erasing data acquired during the tracking, and identifying a signal representative of the surgical tool being in a recording position;
in response of the signal being identified, recording data from the tracking of the tool.

13. The system according to claim 1, wherein recording surfacic data from the tracking of the surgical tool includes recording the surfacic data from a continuous movement of the surgical tool on the anatomical feature surface.

14. A system for navigating through a surgical workflow comprising:
a processing unit; and
a non-transitory computer-readable memory communicatively coupled to the processing unit and comprising computer-readable program instructions executable by the processing unit for:
tracking at least one surgical tool configured for recording points on an anatomical feature surface as the surgical tool contacts the anatomical feature surface, in a first moment of the surgical workflow;
identifying from the tracking a trigger orientation;
in response to the trigger orientation being identified, switching the surgical workflow to a second moment thereof; and
in response to said switching the tracking to the second moment, tracking the at least one surgical tool in accordance with the second moment of the surgical workflow.

15. The system according to claim 14, wherein switching to the second moment of the surgical workflow includes switching to a moment of the surgical workflow that is before the first moment.

16. The system according to claim 15, wherein switching to the second moment of the surgical workflow includes deleting at least some of the points recorded in the first moment.

17. The system according to claim 15, wherein switching to the second moment of the surgical workflow includes prolonging an action associated with the first moment of the surgical workflow.

18. The system according to claim 14, wherein the trigger orientation has the tool pointing substantially upward.

19. The system according to claim 1, including defining a referential system for the anatomical surface feature prior to said tracking.

20. The system according to claim 1, including retrieving the trigger orientation programmed in the non-transitory computer-readable memory.
